(19) United States
(12) Patent Application Publication
(10) Pub. No.: US 2025/0143290 A1
(43) Pub. Date: May 8, 2025

(54) AGROCHEMICAL COMPOSITION COMPRISING AT LEAST ONE AGROCHEMICAL ACTIVE COMPONENT AND A SOLVENT

(71) Applicant: MINAGRO SRL, Louvain-la-Neuve (BE)

(72) Inventors: Arnold DE MAERE, Louvain-la-Neuve (BE); Emmanuel PEULENS, Louvain-la-Neuve (BE)

(21) Appl. No.: 18/833,035

(22) PCT Filed: Jan. 30, 2023
(86) PCT No.: PCT/EP2023/052156

§ 371 Date: Jul. 25, 2024

(30) Foreign Application Priority Data
Jan. 31, 2022 (EP) 22154170.9

(51) Int. Cl. A01N 25/02; A01P 3/00; A01P 7/02; A01P 7/04; A01P 9/00; A01P 11/00; A01P 13/00; A01P 21/00; C07D 307/12; C07D 317/22

(52) U.S. Cl. CPC A01N 25/02; A01P 3/00; A01P 7/02; A01P 7/04; A01P 9/00; A01P 11/00; A01P 13/00; A01P 21/00; C07D 307/12; C07D 317/22

(57) ABSTRACT

The current invention relates to an agrochemical composition comprising at least one agrochemical active component and a solvent. Furthermore, the present invention also relates to a method for applying aforementioned agrochemical composition according to the invention to an agricultural substrate, comprising the steps of: emulsifying said agrochemical composition with water to provide an agrochemically applicable liquid, and bringing said agrochemically applicable liquid in contact with said agricultural substrate.

### FIELD OF THE INVENTION

The present invention relates to an agrochemical composition comprising at least one agrochemical active component and a solvent of the formula as described herein below.

In a second aspect, the present invention also relates to a method for controlling phytopathogenic fungi and/or undesired vegetation and/or undesired insect or mite attack and/or for regulating the growth of plants, wherein the agrochemical composition is allowed to act on the respective pests, their environment or on the plants to be protected from the respective pest, on the soil and/or on undesired plants and/or on the crop plants and/or on their environment.

### BACKGROUND

It is very difficult to identify new solvents for agrochemical compositions comprising an agrochemical active component, because such solvents should meet various requirements: good solvency for the agrochemical active components, low phytotoxicity to the crops, low toxicity to humans, low flammability, compatibility to the application equipment of the farmers, low cost, and low environmental impact.

Known solvents used in agrochemical compositions can be toxic to aquatic organisms and can cause a risk due to repeated exposure for humans, such as for example aromatic hydrocarbons (Solvesso 100, Solvesso 100S, Solvesso 150, Solvesso 150ND, Solvesso 200, Solvesso 200ND), cyclic hydrocarbons (Isophorone, cyclohexanone) or amides (N-Methyl Pyrrolidone). These solvents often have the disadvantage that they exhibit significant toxicity to the users exposed to it, being highly volatile and/or having a flashpoint causing them to be considered highly flammable.

In addition, known solvents, such as Solvesso 100 and Solvesso 150, are petroleum-based solvents, comprising aromatics and naphtha, which makes these solvents hardly biodegradable and less sustainable.

Nonetheless, there is still a need for solvents which meet the requirements of today's agrochemical practice to a further extent.

An object of the present invention was to provide a composition comprising an agrochemical active component, which overcomes the above-mentioned disadvantages.

A solvent and a composition comprising a pesticide in said solvent are disclosed in WO2013153030A1.

However, in the composition disclosed in WO2013153030A1 no account has been taken of the origin of the solvent, e.g., biobased or petroleum based. Furthermore, the exemplified solvents are quite polar, therefore having reduced solvent capacity for apolar pesticides. Furthermore, no account has been taken of the biodegradability of the solvent. In addition, no adjuvant properties of the solvent in the composition have been taken into account.

The present invention aims to resolve at least some of the problems and disadvantages mentioned above.

### SUMMARY OF THE INVENTION

The present invention and embodiments thereof serve to provide a solution to one or more of above-mentioned disadvantages. To this end, the present invention relates to an agrochemical composition according to claim 1.

Preferred embodiments of the composition are shown in any of the claims 2 to 14.

In a second aspect, the present invention relates to a method according to claim 15. The method as described herein provides a method for applying aforementioned agrochemical composition to an agricultural substrate.

It is a prime objective of the present invention to overcome abovementioned disadvantages of the prior art by providing an agrochemical composition comprising a solvent that meets at least one of the following criteria:
Fully or partially bio-based,
good solvency for the agrochemical active components,
good adjuvant properties, such as leaf penetration, distribution/spreading and droplet behavior,
low VOC emissions,
high flash point and boiling point,
biodegradable, and
low toxicity profile.

Furthermore, the present invention provides an alternative for known solvents used in agrochemical compositions, such as for example Solvesso 100, which is a solvent toxic to aquatic organisms and which can cause skin dryness or cracking due to repeated exposure for humans, or Isophorone, which is suspected of causing cancer. In addition, Solvesso 100 and Solvesso 150 are petroleum-based solvents, comprising aromatics and naphta, which makes the solvents hardly biodegradable and less sustainable.

### DESCRIPTION OF FIGURES

The following description of the figures of specific embodiments of the invention is merely exemplary in nature and is not intended to limit the present teachings, their application or uses.

FIG. 1 shows a validated absence of phytotoxicity on salads for the control (A), 1% n-butyl THF (B), 1% t-butyl THF (C), 2% n-butyl THF (D), 2% t-butyl THF (E), 5% n-butyl THF (F), 5% t-butyl THF (G), 10% n-butyl THF (H) and 10% t-butyl THF (I), according to an embodiment of the invention.

FIG. 2 shows observations of the adaxial face of the wheat leave with UV light, according to an embodiment of the invention

FIG. 3 shows observations of the adaxial face of the soybean leave with UV light, according to an embodiment of the invention.

FIG. 4 shows observations of the adaxial and abaxial face of the wheat leave with UV light, according to an embodiment of the invention.

FIG. 5 shows observations of the soybean leaves with UV light, according to an embodiment of the invention.

FIG. 6 shows observations of the adaxial and abaxial face of the wheat leave with UV light, according to an embodiment of the invention.

### DETAILED DESCRIPTION OF THE INVENTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

The expressions “2-(butoxymetyl) THF”, “2-(butoxymetyl)tetrahydrofuraan”, “butyl THF”, “n-butyl THF” and “N-but”, as used in this text, are synonyms and refer to the chemical compound with linear formula C9H18O2 and CAS number 19114-88-6.

The expressions “tertbutyl THF”, “terbutyl THF”, “2-(tert-Butoxymethyl)tetrahydrofuran”, “t-butyl THF” and “T-but”, as used in this text, are synonyms and refer to the chemical compound with linear formula C9H18O2 and CAS number 61590-76-9.

The expressions “n-butyl glycasol”, “butyl glycasol”, and “1,3-Dioxolane,4-(butoxymethyl)-2,2-dimethyl-”, as used in this text, are synonyms and refer to the chemical compound with CAS number 99851-17-9.

The expressions “terbutyl glycasol”, “tertbutyl glycasol”, “t-butyl glycasol” and “1,3-Dioxolane,4-[(1,1-dimethylethoxy)methyl]-2,2-dimethyl-”, as used in this text, are synonyms and refer to the chemical compound with CAS number 122977-52-0.

The expressions “n-butyl glycamal”, “butyl glycamal” and “1,3-Dioxolane, 4-(butoxymethyl)-”, as used in this text, are synonyms and refer to the chemical compound with CAS number 19921-27-8.

The expressions “terbutyl glycamal”, “tertbutyl glycamal”, “t-butyl glycamal” and “1,3-Dioxolane, 4-[(1,1-dimethylethoxy)methyl]-”, as used in this text, are synonyms and refer to the chemical compound with CAS number 2411580-72-6.

The expression “bio-based”, as used in the text, refers to materials that are made from renewable raw materials such as but not limited to starch, sugar, cellulose, hemicellulose, lactic acid, proteins or via micro-organisms.

The expression “biodegradable”, as used in the text, refers to the ability of materials to get disintegrated (decomposed) by the action of micro-organisms such as bacteria or fungi biological (with or without oxygen) while getting assimilated into the natural environment.

In a first aspect, the invention relates to an agrochemical composition comprising at least one agrochemical active component and a solvent of the formula (A):

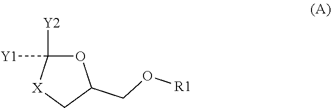

wherein
   X is chosen from O or CH₂;
   Y1 and Y2 are independently of one another chosen from H or a linear or branched C1-C5 alkyl, or Y1 and Y2 together are a keto-group; and
   R1 is chosen from a linear or branched C1-C20 alkyl.

In an embodiment of the invention, the invention relates to an agrochemical composition comprising at least one agrochemical active component and a solvent of the formula (A), wherein
X is chosen from O or CH₂;
Y1 is chosen from H or a linear or branched C1-C5 alkyl, wherein i is a single bond or Y1 is O, wherein i is a double bond and Y2 is removed from the formula;
Y2 is chosen from H or a linear or branched C1-C5 alkyl or Y2 is removed from the formula if Y1 is O; and
R1 is chosen from a linear or branched C1-C20 alkyl.

In an embodiment of the invention, the invention relates to an agrochemical composition comprising at least one agrochemical active component and a solvent of the formula (A), wherein
X is chosen from O or CH₂;
Y1 is chosen from H or a linear or branched C1-C5 alkyl, or Y1 and Y2 together form a keto-group;
Y2 is chosen from H or a linear or branched C1-C5 alkyl, or Y1 and Y2 together form a keto-group; and
R1 is chosen from a linear or branched C1-C20 alkyl.

In an embodiment of the invention, the invention relates to an agrochemical composition comprising at least one agrochemical active component and a solvent of the formula (A), wherein
X is chosen from O or CH₂;
Y1 is chosen from H, O or a linear or branched C1-C5 alkyl, wherein i is a double bond if Y1 is O and i is a single bond if Y1 is H or a linear or branched C1-C5 alkyl, wherein a keto group is formed if Y1 is O;
Y2 is chosen from H or a linear or branched C1-C5 alkyl or Y2 is removed from the formula if Y1 is O; and
R1 is chosen from a linear or branched C1-C20 alkyl.

In a preferred embodiment of the invention, X is CH₂. In another embodiment of the invention, X is O.

In a preferred embodiment of the invention, Y1 and Y2 are independently of one another chosen from H or a linear or branched C1-C5 alkyl. In this embodiment of the invention, Y1 is chosen from H or a linear or branched C1-C5 alkyl and | is a single bond. In a more preferred embodiment of the invention, Y1 and Y2 are H.

In another embodiment of the invention Y1 and Y2 together are a keto-group. In this embodiment of the invention, Y1 is O and i is a double bond, wherein a keto group is formed.

In a preferred embodiment of the invention, R1 is chosen from a linear or branched C1-C12 alkyl, preferably from a linear or branched C1-C8 alkyl, more preferably from a linear or branched C1-C4 alkyl. In a more preferred embodiment of the invention, R1 is butyl, preferably R1 is n-butyl or tert-butyl, more preferably R1 is tert-butyl.

Specifically suitable solvents of the formula (A) are those of the formulae A1 and A2, with the formula A1 being most preferred. In another embodiment the solvent of formula A2 is preferred.

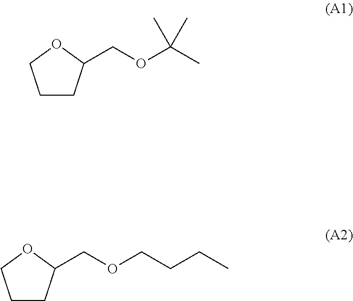

The solvent of the formula (A) usually has a solubility in water at 20° C. of at least 1.0% by weight, preferably of at least 5% by weight, especially preferably of at least 10% by weight.

In an embodiment of the invention, said agrochemical composition comprises said solvent of the formula (A) in an amount of 0.1 to 99% by weight, preferably 3 to 80% by weight, more preferably 10 to 70% by weight.

The agrochemical composition comprises at least one agrochemical active component in dissolved form. Preferably, the agrochemical active component is dissolved in a phase which comprises the solvent of the formula (A). For example, the agrochemical active component is dissolved in a homogenous solution, which comprises the solvent of the formula (A) (e.g. a SL formulation); or the agrochemical active component is dissolved in an emulsified phase, which comprises the solvent of the formula (A) (e.g. droplets containing the agrochemical active component in dissolved form and the solvent of the formula (A), wherein the droplets are emulsified in a continuous phase); or the agrochemical active component is dissolved in a continuous phase of a dispersion, where the continuous phase comprises the solvent of the formula (A) (e.g. an oil-in-water emulsion).

More preferably, the agrochemical composition comprises at least one agrochemical active component and a solvent of the formula (A) in form of a homogenous solution. Further components (e.g. further solvents, auxiliaries, further agrochemical active components) may be present in the same phase together with the solvent of the formula (A).

In a preferred embodiment of the invention, the solvent of the formula (A) has an adjuvant property. Adjuvant property refers to any property of the solvent which increases the efficacy or potency of the agrochemical composition or the agrochemical active component. Adjuvant properties can include but are not limited to penetration, distribution/coverage and droplet behavior.

The expression “agrochemical active component” refers to at least one active substance selected from the list of herbicides, fungicides, insecticides, nematicides, algicides, molluscicides, miticides, rodenticides, safeners, plant growth regulators, biostimulants or a combination thereof. Preferably, the agrochemical active component is chosen from the list of herbicides, fungicides, insecticides and biostimulants. Mixtures of agrochemical active components of two or more of the abovementioned classes may also be used. The skilled worker is familiar with such pesticides, which can be found, for example, in Pesticide Manual, 14th Ed. (2006), The British Crop Protection Council, London.

In a preferred embodiment of the invention, said agrochemical active component is an insecticide chosen from the classes of carbamates, organophosphates, organochlorine insecticides, phenylpyrazoles, pyrethroids, neonicotinoids, spinosins, avermectins, milbemycins, juvenile hormone analogs, alkyl halides, organotin compounds, nereistoxin analogs, benzoylureas, diacylhydrazines, METI acaricides, and insecticides such as chloropicrin, pymetrozine, flonicamid, clofentezine, hexythiazox, etoxazole, diafenthiuron, propargite, tetradifon, chlorfenapyr, DNOC, buprofezin, cyromazine, amitraz, hydramethylnon, acequinocyl, fluacrypyrim, rotenon, anthranilic diamides, or their derivatives. In a more preferred embodiment of the invention, said agrochemical active component is an insecticide chosen from the classes of juvenile hormone analogs, neonicotinoids, pyrethroids and anthranilic diamides. In an even more preferred embodiment of the invention, said agrochemical active component is an insecticide chosen from the list of pyriproxyfen, imidacloprid, cypermethrin, chlorantraniliproleor a mixture thereof.

In a preferred embodiment of the invention, said agrochemical active component is a fungicide chosen from the list of dinitroanilines, allylamines, anilinopyrimidines, antibiotics, aromatic hydrocarbons, benzenesulfonamides, benzimidazoles, benzoisothiazoles, benzophenones, benzothiadiazoles, benzotriazines, benzylcarbamates, carbamates, carboxamides, carboxylic acid amides, chloronitriles, cyanoacetamide oximes, cyanoimidazoles, cyclopropanecarboxamides, dicarboximides, dihydrodioxazines, dinitrophenyl crotonates, dithiocarbamates, dithiolanes, ethylphosphonates, ethylaminothiazolecarboxamides, guanidines, hydroxy(2-amino)pyrimidines, hydroxyanilides, imidazoles, imidazolinones, inorganic substances, isobenzofuranones, methoxyacrylates, methoxycarbamates, morpholines, N-phenylcarbamates, oxazolidinediones, oximinoacetates, oximinoacetamides, peptidylpyrimidine nucleosides, phenylacetamides, phenylamides, phenylpyrroles, phenylureas, phosphonates, phosphoro-thiolates, phthalamic acids, phthalimides, piperazines, piperidines, propionamides, pyridazinones, pyridines, pyridinylmethylbenzamides, pyrimidinamines, pyrimidines, pyrimidinonehydrazones, pyrroloquinolinones, quinazolinones, quinolines, quinones, sulfamides, sulfamoyltriazoles, strobilurins, thiazolecarboxamides, thiocarbamates, thiophanate, thiophene-carboxamides, toluamides, triphenyltin compounds, triazines, triazoles, triazolinthiones and morpholines. In a more preferred embodiment of the invention, said agrochemical active component is a fungicide chosen from the classes of strobilurins, triazoles, triazolinthiones and morpholines.

In an even more preferred embodiment of the invention, said agrochemical active component is a fungicide chosen from the list of azoxystrobin, metconazole, difenoconazole, tebuconazole, prothioconazole and spiroxamine or a mixture thereof.

In a preferred embodiment of the invention, said agrochemical active component is an herbicide chosen from the list of acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ethers, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyndinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas, anilide herbicides. In a more preferred embodiment of the invention, said agrochemical active component is an herbicide chosen from the classes of aryloxyphenoxypropionates, anilide herbicides, sulfonylureas, pyridines and cyclohexanediones. In an even more preferred embodiment of the invention, said agrochemical active component is an herbicide chosen from the list of quizalofop-p-ethyl, diflufenican, phenmidipham, nicosulfuron, fluroxypyr and clethodim or a mixture thereof.

In a preferred embodiment of the invention, said agrochemical active component is a biostimulant which is defined as substance(s) and/or microorganisms whose function when applied to plants or the rhizosphere is to stimulate natural processes to enhance/improve nutrient uptake, nutrient efficiency, tolerance to abiotic stress, and/or crop quality. Biostimulants can also include complex mixtures such as plant extracts, fermentation solutions, or food processing byproducts.

In another embodiment of the invention, the agrochemical active component is chosen from the list of germicides, antibiotics, antibacterial agents, antiviral agents, antifungal agents, antiprotozoal agents, antiparasitic agents, or a combination thereof.

The agrochemical composition according to the invention can also comprise further agrochemical active components. The further agrochemical active components can be present in dissolved, suspended and/or emulsified form. In a further embodiment, at least one agrochemical active component is suspended to at least 90% by weight, based on the agrochemical active component, in the solvent system in the form of solid particles. If the agrochemical composition comprises at least two agrochemical active components, at least one agrochemical active component may be dissolved to at least 90% by weight in the solvent system. Preferably, the pesticide is suspended to at least 95% by weight, especially preferably to at least 98% by weight, in the solvent system.

The agrochemical composition according to the invention usually comprises from 0.1 to 70% by weight of pesticide, preferably from 1 to 50%, in particular from 3 to 30% by weight, based on the agrochemical composition.

In an embodiment of the invention, the agrochemical composition comprises formulation auxiliaries, the choice of the auxiliaries usually depending on the specific embodiment and/or the active substance. Examples for suitable auxiliaries are additional solvents, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable additional solvents which may be present in the agrochemical composition in addition to the solvent of the formula (A) are organic solvents. Suitable additional solvents are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydro-naphthalene, alkylated naphthalenes; alcohols, e.g. ethanol, propanol, butanol, benzyl alcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

In principle, it is also possible to use solvent mixtures. It is preferred to add up to 40% by weight, preferably up to 20% by weight, and in particular up to 5 wt %, of additional solvents to the agrochemical composition according to the invention, in each case based on the agrochemical composition. In another preferred form the agrochemical composition is essentially free of additional solvents. The agrochemical composition may be essentially free of additional solvents like amides based on ketocarboxylic acids, esters based on ketocarboxylic acids, monopropylenglycol esters, ester of hydroxycarboxylic acids, C8-C12 fatty acid dialkyl amides, or dialkylamides based on oleic or linoleic acid. In a preferred embodiment, the solvent mixture is essentially free of additional solvents not according to formula A.

In another form the agrochemical composition is essentially free of water, for example the agrochemical composition may comprise up to 5 wt %, preferably up to 2 wt %, more preferably up to 0.5 wt % of water.

Suitable solid carriers or fillers are mineral earths, e.g. silicates, silica gels, talc, kaolins, lime-stone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.). The agrochemical composition according to the invention preferably comprises at least one anionic surfactant. The agrochemical composition according to the invention preferably comprises at least one nonionic surfactant. The agrochemical composition according to the invention more preferably comprises at least one anionic surfactant and at least one nonionic surfactant. The agrochemical composition according to the invention may be essentially free of cationic surfactants.

The agrochemical composition according to the invention can comprise various amounts of surfactants. It can comprise from 0.1 to 40% by weight, preferably from 1 to 30 and in particular from 2 to 20% by weight total amount of surfactant, based on the total amount of the agrochemical composition.

Suitable anionic surfactants are alkali metal, earth alkali metal or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates. Preferred anionic surfactants are sulfonates of ethoxylated arylphenols, in particular phosphated or sulfated di- and/or tristyrylphenyl alkoxylates, as are described for example in WO 2007/1 10355, page 3, line 30 to page 5, line 1 1.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidone, vinylalcohols, or vinylacetate. The term “nonionic surfactant” does usually not relate to the solvent of the formula (A). Typically, the solvent of the formula (A) is not suitable as nonionic surfactant (probably because it does not comprise a polar and a nonpolar residue as usual for nonionic surfactants).

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids. Suitable colorants (e.g. in red, blue, or green) are pigments of low water solubility and water-soluble dyes. Examples are inorganic colorants (e.g. iron oxide, titan oxide, iron hexacyanofer-rate) and organic colorants (e.g. alizarin-, azo- and phthalocyanine colorants).

Suitable tackifiers or binders are polyvinylpyrrolidone, polyvinylacetates, polyvinyl alcohols, pol-yacrylates, biological or synthetic waxes, and cellulose ethers.

In a second aspect, the invention relates to a method for applying aforementioned agrochemical composition according to the invention to an agricultural substrate, comprising the steps of:
emulsifying said agrochemical composition with water to provide an agrochemically applicable liquid, and
bringing said agrochemically applicable liquid in contact with said agricultural substrate.

In a preferred embodiment of the invention, the method controls phytopathogenic fungi and/or undesired vegetation and/or undesired insect or mite attack and/or for regulating the growth of plants, wherein the aforementioned composition is allowed to act on the respective pests, their environment or on the plants to be protected from the respective pest, on the soil and/or on undesired plants and/or on the crop plants and/or on their environment.

Advantages of the present invention are that an agrochemical composition is provided comprising a solvent that meets at least one, and preferably multiple, of the following criteria:
fully or partly bio-based,
good solvency for the agrochemical active components,
good adjuvant properties, such as leaf penetration, distribution/spreading and droplet behavior,
low VOC emissions,
high flash point and boiling point,
biodegradable, and
low toxicity and low phytotoxicity.

Furthermore, the present invention provides an alternative for known solvents used in agrochemical compositions, such as for example Solvesso 100, which is a solvent toxic to aquatic organisms and which can cause skin dryness or cracking due to repeated exposure for humans, or Isophorone, which is suspected of causing cancer. In addition, Solvesso 100 and Solvesso 150 are petroleum-based solvents, comprising aromatics and naphta, which makes the solvents hardly biodegradable and less sustainable.

The invention is further described by the following non-limiting examples which further illustrate the invention, and are not intended to, nor should they be interpreted to, limit the scope of the invention.

### EXAMPLES

The present invention will now be further exemplified with reference to the following examples. The present invention is in no way limited to the given examples or to the embodiments presented in the figures.

### Example 1: Hansen Solubility Parameters

Example 1 refers to the Hansen solubility parameters and the Hildebrand solubility parameter for n-butyl THF and t-butyl THF compared to known agrochemical solvents, as shown in table 1. The Hansen solubility parameters are used for predicting if one material will dissolve in another and form a (homogeneous) solution.

Furthermore, solvents with similar Hansen solubility parameters will have similar solubility characteristics, including which agrochemical active components will and which agrochemical active components will not dissolve in the solvents.

TABLE 1

| | Hildebrand | Hansen Solubility | | |
|---|---|---|---|---|
| | (J/cm³) | Parameters (J/cm³) | | |
| Example 1 | δ | δd | δp | δn |
| Solvent of the formula (A) | | | | |
| n-butyl THF | 18.15 | 16.47 | 4.74 | 5.96 |
| t-butyl THF | 17.98 | 16.28 | 4.75 | 5.97 |
| Known agrochemical solvents | | | | |
| 2-Ethylhexyl Lactate | 18.41 | 15.7 | 4.5 | 8.5 |
| (CAS: 6283-86-9) | | | | |
| p-Xylène | 17.90 | 17.6 | 1 | 3.1 |
| (CAS: 106-42-3) | | | | |
| MEK | 19.05 | 16 | 9 | 5.1 |
| (CAS: 78-93-3) | | | | |
| N-Methyl-2-Pyrrolidone | 22.96 | 18 | 12.3 | 7.2 |
| (CAS: 872-50-4) | | | | |
| Ethyl Acetate | 18.15 | 15.8 | 5.3 | 7.2 |
| (CAS: 141-78-6) | | | | |
| Isopropyl Alcohol | 23.58 | 15.8 | 6.1 | 16.4 |
| (CAS: 67-63-0) | | | | |
| n-butyl Alcohol | 14.78 | 13.8 | 2.8 | 4.5 |
| (CAS: 71-36-3) | | | | |
| Cyclohexanone | 19.56 | 17.8 | 6.3 | 5.1 |
| (CAS: 108-94-1) | | | | |
| THFA TetraHydroFurfurylAlcohol | 22.09 | 17.8 | 8.2 | 10.2 |
| (CAS: 97-99-4) | | | | |
| Methyl Acetate | 18.70 | 15.5 | 7.2 | 7.6 |
| (CAS: 79-20-9) | | | | |
| Dimethyl Sulfoxide | 26.68 | 18.4 | 16.4 | 10.2 |
| (CAS: 67-68-5) | | | | |
| Isophorone | 19.94 | 16.6 | 8.2 | 7.4 |
| (CAS: 78-59-1) | | | | |
| Acetophenone | 21.72 | 19.6 | 8.6 | 3.7 |
| (CAS: 98-86-2) | | | | |
| n-butyl acetate | 17.41 | 15.8 | 3.7 | 6.3 |
| (CAS: 123-86-4) | | | | |

### Example 2-4: Theorical Solubility of Agrochemical Active Components in Solvents of the Formula (A)

Example 2 refers to the theoretical solubility of different agrochemical active components in different solvents of the formula (A), calculated using COSMO-RS.

COSMO-RS (short for Conductor like Screening MOdel for Real Solvents) is a quantum chemistry-based equilibrium thermodynamics method with the purpose of predicting chemical potentials u in liquids. It processes the screening charge density σ on the surface of molecules to calculate the chemical potential μ of each species in solution. Perhaps in dilute solution a constant potential must be considered. As an initial step a quantum chemical COSMO calculation for all molecules is performed and the results (e.g. the screening charge density) are stored in a database. In a separate step COSMO-RS uses the stored COSMO results to calculate the chemical potential of the molecules in a liquid solvent or mixture. The resulting chemical potentials are the basis for other thermodynamic equilibrium properties such as activity coefficients, solubility, partition coefficients, vapor pressure and free energy of solvation.

Table 2 shows the calculated theoretical solubility of sixteen different agrochemical active components in six different solvents of the formula (A) in g/L.

Comparative example 3 refers to compositions with ten different agrochemical active components currently available on the market, as shown in table 3.

Comparative example 4 refers to the calculated theoretical solubility of sixteen different agrochemical active components in butyl levulinate (CAS: 2052-15-5), as shown in table 4.

The results show that the six solvents of the formula (A) are suitable alternatives to isophorone and aromatic solvents (Solvesso). Comparison also shows n-butyl THF and t-butyl THF have a similar or higher solubility for all sixteen agrochemical active components compared to butyl levulinate.

TABLE 2

| | n-butyl | t-butyl | | | | |
|---|---|---|---|---|---|---|
| Example 2 | THF | THF | (1) | (2) | (3) | (4) |
| Quizalofop-P-ethyl | 68.58 | 99.29 | 21.34 | 30.41 | 63.26 | 25.12 |
| (CAS: 100646-51-3) | | | | | | |
| Diflufenican | 289.42 | 388.7 | 119.66 | 92.27 | 221.16 | 76.69 |
| (CAS: 83164-33-4) | | | | | | |
| Phenmidipham | 215.67 | 306.39 | 101.93 | 46.79 | 145.53 | 38.86 |
| (CAS: 13684-63-4) | | | | | | |
| Nicosulfuron | 14.61 | 62.19 | 39.66 | 0.5 | 7.39 | 0.37 |
| (CAS: 111991-09-4) | | | | | | |
| Fluroxypyr | 739.51 | 824.96 | 710.95 | 433.37 | 644.04 | 440.58 |
| (CAS: 69377-81-7) | | | | | | |
| Clethodim | 194.56 | 288.95 | 42.97 | 18.03 | 57.47 | 21.73 |
| (CAS: 99129-21-2) | | | | | | |
| Azoxystrobin | 28.99 | 52.65 | 18.3 | 10.1 | 37.13 | 7.49 |
| (CAS: 131860-33-8) | | | | | | |
| Metconazole | 28.77 | 55.94 | 10.77 | 5.49 | 12.3 | 5.08 |
| (CAS: 125116-23-6) | | | | | | |
| Difenoconazole | 10.58 | 18.7 | 4.05 | 3.21 | 7.49 | 2.61 |
| (CAS: 119446-68-3) | | | | | | |
| Tebuconazole | 152.68 | 232.9 | 52.6 | 27.84 | 66.21 | 26.59 |
| (CAS: 107534-96-3) | | | | | | |
| Prothioconazole | 123.3 | 235.02 | 49.09 | 5.63 | 39.52 | 4.48 |
| (CAS: 178928-70-6) | | | | | | |
| Sprioxamine | 58.44 | 57.99 | 8.33 | 44.17 | 31.16 | 45.71 |
| (CAS: 118134-30-8) | | | | | | |
| Pyriproxyfen | 138.5 | 167.64 | 19.29 | 83.39 | 120.76 | 71.99 |
| (CAS: 95737-68-1) | | | | | | |
| Imidacloprid | 16.05 | 31.81 | 104.32 | 5.37 | 24.65 | 4.2 |
| (CAS: 138261-41-3) | | | | | | |
| Cypermethrin | 484.99 | 528.45 | 56.96 | 353.52 | 440.42 | 326.4 |
| (CAS: 52315-07-8) | | | | | | |
| Chlorantraniliprole | 3.39 | 6.5 | 1.79 | 1.08 | 2.89 | 0.84 |
| (CAS: 500008-45-7) | | | | | | |

Wherein:
(1) = n-butyl glycasol (CAS: 99851-17-9)
(2) = t-butyl glycasol (CAS: 122977-52-0)
(3) = n-butyl glycamal (CAS: 19921-27-8)
(4) = t-butyl glycamal (CAS: 2411580-72-6)

TABLE 3

| Comparative | Products on the market | |
|---|---|---|
| example 3 | g/L | Solvent |
| Quizalofop-P-ethyl | 100 | N-Methyl Pyrrolidone |
| (CAS: 100646-51-3) | | |
| Phenmidipham | 160 | Isophorone |
| (CAS: 13684-63-4) | | |
| Fluroxypyr | 200 | Solvesso 100 |
| (CAS: 69377-81-7) | | |
| Clethodim | 240 | Solvesso 100 |
| (CAS: 99129-21-2) | | |
| Metconazole | 60 | — |
| (CAS: 125116-23-6) | | |
| Difenoconazole | 250 | Solvesso 150 + NMP |
| (CAS: 119446-68-3) | | |
| Tebuconazole | 250 | N,N-Dimethyl decanamide |
| (CAS: 107534-96-3) | | |
| Prothioconazole | 250 | N,N-Dimethyl decanamide |
| (CAS: 178928-70-6) | | |
| Pyriproxyfen | 100 | Solvesso 150 |
| (CAS: 95737-68-1) | | |
| Cypermethrin | 500 | Solvesso 100 |
| (CAS: 52315-07-8) | | |

TABLE 4

| Comparative | Butyl levulinate |
|---|---|
| example 4 | (CAS: 2052-15-5) |
| Quizalofop-P-ethyl | 63.43 |
| (CAS: 100646-51-3) | |
| Diflufenican | 237.08 |
| (CAS: 83164-33-4) | |
| Phenmidipham | 155.15 |
| (CAS: 13684-63-4) | |
| Nicosulfuron | 12.09 |
| (CAS: 111991-09-4) | |
| Fluroxypyr | 593.26 |
| (CAS: 69377-81-7) | |
| Clethodim | 41.45 |
| (CAS: 99129-21-2) | |
| Azoxystrobin | 47.64 |
| (CAS: 131860-33-8) | |
| Metconazole | 11.02 |
| (CAS: 125116-23-6) | |
| Difenoconazole | 7.96 |
| (CAS: 119446-68-3) | |
| Tebuconazole | 58.81 |
| (CAS: 107534-96-3) | |
| Prothioconazole | 50.86 |
| (CAS: 178928-70-6) | |
| Sprioxamine | 18.21 |
| (CAS: 118134-30-8) | |
| Pyriproxyfen | 115.99 |
| (CAS: 95737-68-1) | |
| Imidacloprid | 36.03 |
| (CAS: 138261-41-3) | |
| Cypermethrin | 403.53 |
| (CAS: 52315-07-8) | |
| Chlorantraniliprole | 3.23 |
| (CAS: 500008-45-7) | |

### Example 5-6: Experimental Solubility

Example 5 and example 6 refer to the experimental maximum solubility of different agrochemical active components in different solvents of the formula (A), calculated using COSMO-RS.

Saturated solutions of agrochemical active components in n-butyl THF and t-butyl THF were prepared under MT 181. Said saturated solutions were subsequently analyzed by HPLC to determine the agrochemical active component concentration. The samples were centrifuged and filtered twice through 1.6 and 0.45 μm filters to remove any extraneous material prior to analysis. Results are quoted in terms of pure active ingredient (g/L) and given in table 5 and table 6.

For each combination of agrochemical active component, a calibration curve was established to plot the instrumental response, e.g., the analytical signal, changes with the concentration of the agrochemical active component.

Table 5 shows the experimental agrochemical active component solubility in saturation (g/L) of nine different agrochemical active components in t-butyl THF compared to compositions which are currently available on the market.

Table 6 shows the experimental agrochemical active component solubility in saturation (g/L) of four different agrochemical active components in n-butyl THF compared to compositions which are currently available on the market.

The results validate n-butyl THF and t-butyl THF as a greener alternative for aromatic solvents, such as Solvesso 100 and Solvesso 150. Especially for the dissolution of triazole fungicides, such as metconazole, difenoconazole, tebuconazole and prothioconazole.

TABLE 5

| | | | t-butyl THF | |
|---|---|---|---|---|
| | Products on the market | | | Solubility in |
| Example 5 | g/L | Solvent | COMSO-RS | saturation |
| Phenmidipham | 160 | Isophorone | 306.39 | 37.11 |
| (CAS: 13684-63-4) | | | | |
| Fluroxypyr | 200 | Solvesso 100 | 824.96 | 537.29 |
| (CAS: 69377-81-7) | | | | |
| Clethodim | 240 | Solvesso 100 | 288.95 | 1478.63 |
| (CAS: 99129-21-2) | | | | |
| Metconazole | 60 | — | 55.94 | 210.45 |
| (CAS: 125116-23-6) | | | | |
| Difenoconazole | 250 | N-Methyl | 18.7 | 342.72 |
| (CAS: 119446-68-3) | | Pyrrolidone | | |
| Tebuconazole | 250 | N,N-Dimethyl | 232.9 | 187.65 |
| (CAS: 107534-96-3) | | decanamide | | |
| Prothioconazole | 250 | N,N-Dimethyl | 235.02 | 341.15 |
| (CAS: 178928-70-6) | | decanamide | | |
| Pyriproxyfen | 100 | Solvesso 150 | 167.64 | 630.90 |
| (CAS: 95737-68-1) | | | | |
| Cypermethrin | 500 | Solvesso 100 | 528.45 | 1054.95 |
| (CAS: 52315-07-8) | | | | |

TABLE 6

| | | | n-butyl THF | |
|---|---|---|---|---|
| | Products on the market | | | Solubility in |
| Example 6 | g/L | Solvent | COMSO-RS | saturation |
| Phenmidipham | 160 | Isophorone | 215.67 | 35.66 |
| (CAS: 13684-63-4) | | | | |
| Fluroxypyr | 200 | Solvesso 100 | 739.51 | 546.25 |
| (CAS: 69377-81-7) | | | | |
| Tebuconazole | 250 | N,N-Dimethyl | 152.68 | 181.24 |
| (CAS: 107534-96-3) | | decanamide | | |
| Cypermethrin | 500 | Solvesso 100 | 484.99 | 1068.58 |
| (CAS: 52315-07-8) | | | | |

### Example 7: Phytotoxicity Study of n-Butyl THF and t-Butyl THF

Example 7 refers to a study of the potential phytotoxicity of two solvents, n-butyl-THF and t-butyl THF, on Lactuca sativa.

The aim of this experiment is to determine if these solvents can cause phytotoxicity on lettuce at different concentrations.

Lettuce of the Lucrecia RZ variety was sown in potting soil and transplanted into 3L pots. For each of the solvents, four concentrations were tested: 1%, 2%, 5% and 10%. A control (water) was also performed. Once the 10-leaf stage was reached by the salads, they were sprayed until saturation with the solvents. Fifteen replicates per treatment were conducted. A monitoring of the appearance of potential spots due to phytotoxicity is carried out 5 days and 10 days after spraying.

None of the concentrations tested showed stains potentially related to phytotoxicities. Only a few stains related to possible wounds on older leaves were found, regardless of the treatment (solvent or control) and the concentration. They are therefore not to be taken into account for any phytotoxicity.

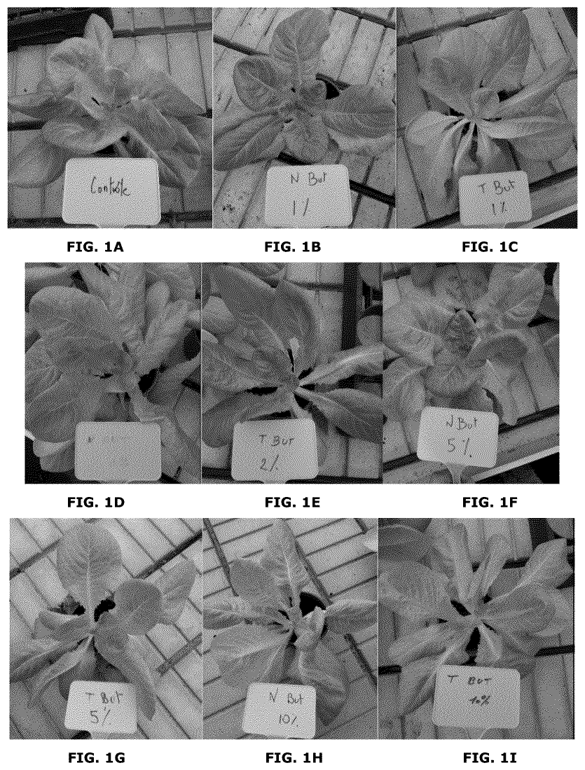

To better exemplify reference is made to FIG. 1, which shows a validated absence of phytotoxicity on salads for the control (A), 1% n-butyl THF (B), 1% t-butyl THF (C), 2% n-butyl THF (D), 2% t-butyl THF (E), 5% n-butyl THF (F), 5% t-butyl THF (G), 10% n-butyl THF (H) and 10% t-butyl THF (I), according to an embodiment of the invention.

### Example 8: Evaluation of Droplet Behavior (Adjuvant Property)

Example 8 refers to an evaluation of n-butyl THF and t-butyl THF according to their ability to affect the behavior of an agrochemical formulation, more specifically their ability to affect droplet behavior. Soybean and wheat leaves were used as biological surfaces for the discrimination of the droplet behavior, each being tested at three doses (1%, 2% and 5%) in three replicates in order to select the most appropriate one. The characteristic of wheat and soybean leaves is that their surfaces are hydrophobic (high for wheat and medium for soybean), which is not compatible with good water retention or, in general, product retention.

A 1 μL droplet of product formulation with fluorescent dye was deposited at the leaf surface (adaxial face). Subsequently, photos were taken for 30 minutes in order to follow the evolution of the droplet.

Table 7 shows the visualization of the behavior and drying of droplet formulations at the surface of the wheat leaf fragment.

Table 8 shows the visualization of the behavior and drying of droplet formulations at the surface of the soybean leaf fragment.

The criteria for evaluating the behavior of a droplet of formulation on a leaf or leaf fragment is as follows:

### Retention of Formulation on Leaf (Arbitrary Scale):

0.5: the droplets stick after more than 10 attempts
1: the droplets stick after 5 to 10 attempts
2: the droplets stick after 2 to 4 attempts
3: the droplets stick immediately
   Shape of the Drop Just after Deposit (Arbitrary Scale)
1: drop round (very little surface contact)
2: light spread
3: moderate spread
4: high spread
5: very high spread

### Shape of the Drop During Drying (Arbitrary Scale)

1: drop round (very little surface contact)
2: light spread
3: moderate spread
4: high spread
5: very high spread

TABLE 7

| | | Drying | Shape of the | Shape of the | |
|---|---|---|---|---|---|
| | | time | drop just | drop during | |
| | Retention | (Min) | after deposit | drying | Total |
| n-butyl THF | 3 | 23′ | 2 | 2 | 7 |
| (1%) | | | | | |
| n-butyl THF | 3 | 18′25″ | 4 | 4 | 11 |
| (2%) | | | | | |
| n-butyl THF | 3 | 6′ | 5 | 5 | 13 |
| (5%) | | | | | |
| t-butyl THF | 3 | 20′ | 1 | 1 | 5 |
| (1%) | | | | | |
| t-butyl THF | 3 | 24′25″ | 1 | 1 | 5 |
| (2%) | | | | | |
| t-butyl THF | 3 | 22′30″ | 3-4 | 3-4 | 10 |
| (5%) | | | | | |

TABLE 8

| | | Drying | Shape of the | Shape of the | |
|---|---|---|---|---|---|
| | | time | drop just | drop during | |
| | Retention | (min) | after deposit | drying | Total |
| n-butyl THF | 3 | 10′20″ | 2 | 2 | 7 |
| (1%) | | | | | |
| n-butyl THF | 3 | 7′30″ | 2-3 | 2-3 | 8 |
| (2%) | | | | | |
| n-butyl THF | 3 | 8′15 | 4 | 4 | 11 |
| (5%) | | | | | |
| t-butyl THF | 3 | 12′50″ | 1 | 1 | 5 |
| (1%) | | | | | |
| t-butyl THF | 3 | 11′05″ | 1 | 2 | 6 |
| (2%) | | | | | |
| t-butyl THF | 3 | 9′ | 2 | 2 | 7 |
| (5%) | | | | | |

The results show that in every replicate, the droplets stick immediately, which indicates that both n-butyl THF and t-butyl THF improve retention on leaves with hydrophobic surfaces.

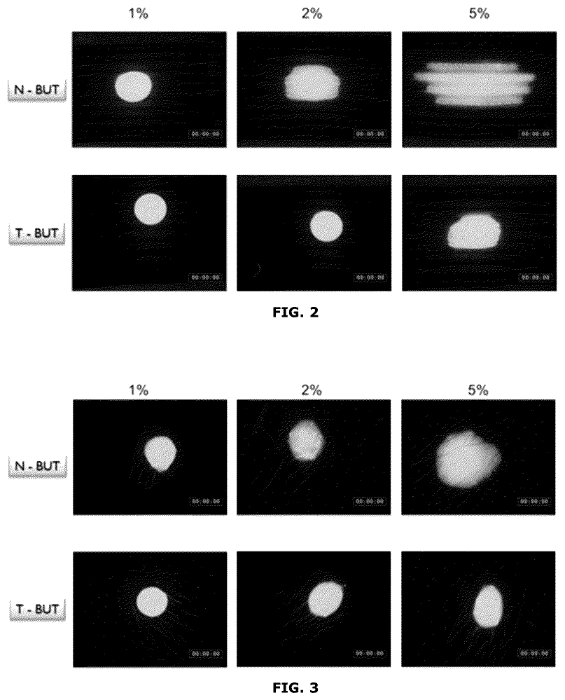

To better exemplify reference is made to FIG. 2, which shows observations of the adaxial face of the wheat leave with UV light and FIG. 3, which shows observations of the adaxial face of the soybean leave with UV light.

### Example 9: Evaluation of the Distribution and Coverage (Adjuvant Property)

Example 9 refers to an evaluation of n-butyl THF and t-butyl THF according to their ability to affect the behavior of an agrochemical formulation, more specifically their ability to affect distribution and coverage. Soybean and wheat leaves were used as biological surfaces for the discrimination of the droplet behavior, each being tested at 2% in two replicates in order to select the most appropriate one. The advantage of wheat and soybean leaves is that their surfaces are hydrophobic (high for wheat and medium for soybean), which is not compatible with good water retention or, in general, product retention.

The product formulation with blue, fluorescent dye was used in a tracksprayer treatment with a TurboTwinjet nozzle. The leaves were dried for 15 hours at room temperature in the dark. Subsequently, the leaves were observed under an epifluorescence microscope.

Table 9 shows the spray distribution and coverage of the formulations on wheat and soybean leaves. The criteria for evaluating the distribution and coverage of formulations (applied at 2%) on a leaf or leaf fragment is as follows:

The number of impacts per leaf (arbitrary scale):
1: very low
2: low
3: moderate
4: high
5: very high

### The Size and Spreading of Impacts (Arbitrary Scale)

1: very low
2: low
3: moderate
4: high
5: very high

### Estimate of the Area Covered by the Formulations (%)

TABLE 9

| | | Number | | | Estimate of |
|---|---|---|---|---|---|
| | | of | | | the area |
| | | impacts | Size/spreading | | covered by the |
| Leaves | Retention | per leaf | of impacts | Total | formulations |
| Wheat | Control | 0 | 0 | 0 | 0% |
| | n-butyl | 3 | 1-4 | 5.5 | 21% |
| | THF | | | | |
| | t-butyl | 3 | 1-2 | 4.5 | 14% |
| | THF | | | | |
| Soybean | Control | 0 | 0 | 0 | 0% |
| | n-butyl | 4 | 1-4 | 6.5 | 46% |
| | THF | | | | |
| | t-butyl | 4 | 1-4 | 6.5 | 34% |
| | THF | | | | |

The results show a good retention for every replicate. The large number and size of impacts indicates good coverage of both formulations on wheat and soybean leaves which have hydrophobic surfaces. It is further shown that n-butyl THF appears to provide better coverage than t-butyl THF.

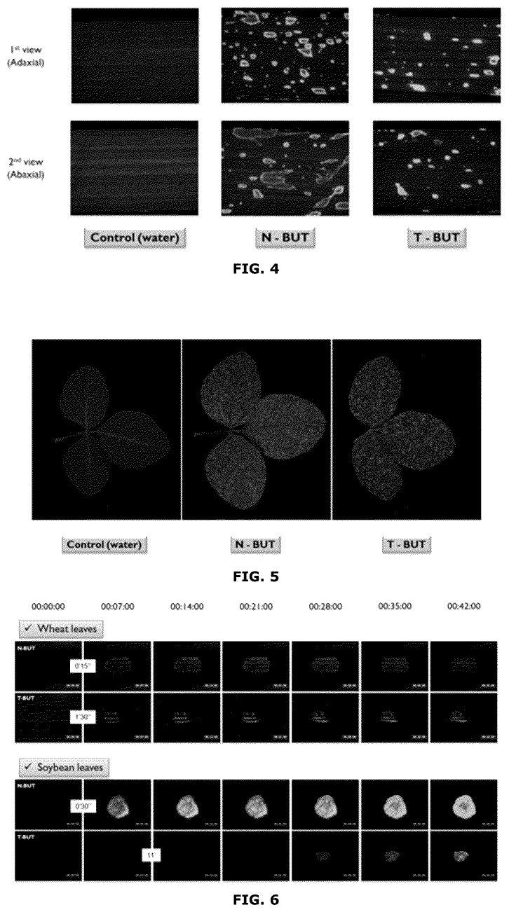

To better exemplify reference is made to FIG. 4, which shows observations of the adaxial and abaxial face of the wheat leave with UV light and FIG. 5, which shows observations of the soybean leaves with UV light.

### Example 10: Driving Force (Adjuvant Property)

Example 10 refers to an evaluation of n-butyl THF and t-butyl THF according to their ability to affect the behavior of an agrochemical formulation, more specifically their ability to affect penetration. Soybean and wheat leaves were used as biological surfaces for the discrimination of the droplet behavior, each being tested at 2% in three replicates in order to select the most appropriate one. The characteristic of wheat and soybean leaves is that their surfaces are hydrophobic (high for wheat and medium for soybean), which is not compatible with good water retention or, in general, product retention.

A 1 μL droplet of product formulation with fluorescent dye was deposited at the leaf surface (adaxial face). Subsequently, photos were taken every 15 seconds for 45 minutes in order to follow the evolution of the droplet at 20° C.

Table 10 shows the penetration driving force of the formulations on wheat and soybean tissues. The criteria for evaluating the penetration strength of formulations on a leaf or leaf fragment is as follows:

### Penetration in the Leaf (Arbitrary Scale):

1: no
2: yes

### Mean Penetration Time of the Formulation Inside Leaf

### Intensity of the Penetration Strength

1: very light
2: light
3: medium
4: high

TABLE 10

| | | | Penetration | Intensity of | |
|---|---|---|---|---|---|
| | | | time | the penetra- | |
| Leaves | Retention | Penetration | (mean) | tion strength | Total |
| Wheat | Control | 1 | — | 0 | 1 |
| | n-butyl | 2 | 57″ | 2 | 4 |
| | THF | | | | |
| | t-butyl | 2 | 60″ | 2 | 4 |
| | THF | | | | |
| Soybean | Control | 1 | — | 0 | 1 |
| | n-butyl | 2 | 40″ | 4 | 6 |
| | THF | | | | |
| | t-butyl | 2 | 12′50″ | 3 | 5 |
| | THF | | | | |

The results show a good penetration for every replicate. The behavior of n-butyl THF and t-butyl THF appears to be similar on wheat leaves, but the penetration time differs for n-butyl THF and t-butyl THF on soybean leaves.

To better exemplify reference is made to FIG. 6, which shows observations of the adaxial and abaxial face of the wheat leave with UV light.

### Example 11: n-Butyl THF Biodegradability Test

Example 11 refers to a 28-day biodegradability test of n-butyl THF consisting of a manometric respiration tests according to OECD301F.

Biodegradation is the breakdown (mineralization) of an organic substance to carbon dioxide, water, mineral salts and microbial biomass. For measuring biodegradability according OECD 301 F the oxygen consumption due to substrate biodegradation in a closed respirometer is determined. The test duration is 28 days. The vessels contained 250 ml of the inoculated buffered mineral salt medium and the test material (test substance or reference substance) as the sole carbon source. To each test series blanks (without test material) were set up to run in parallel. Incubation was conducted at 22±1° C. in diffuse light and agitation by magnetic stirrers. All analyses were conducted at least in duplicate. Oxygen consumption was continuously measured with a respirometer. The amount of oxygen taken up by the microorganisms during biodegradation (biological oxygen demand; BOD) is compared with the chemical oxygen demand (COD) of the test substance. The percent biodegradation of the test substance is calculated from the BOD in relation to the COD or alternative of the theoretical oxygen demand (ThOD) of the test substance. The test material (92 mg/L) was distributed in a thin layer on an inert carrier (glass fiber filter) and added directly to the aqueous medium.

Table 11 gives the time course of biodegradation of the test sample, reference and toxicity control.

The results show that the test sample (comprising n-butyl THF) is 100% readily biodegradable, with already 91% biodegradability reached on day 18 and 100% biodegradability reached on day 23.

In addition, according the data of the toxicity control the test sample is considered to be non-inhibitory to the microorganisms of the inoculum.

TABLE 11

| | Biodegradation (%) | | |
|---|---|---|---|
| | Test sample | Reference | Toxicity control |
| 0 | 0.0 | 0.0 | 0.0 |
| 7 | 5.6 | 88 | 35 |
| 14 | 77 | 94 | 69 |
| 21 | 96 | 98 | 83 |
| 28 | 105 | 98 | 86 |

It is supposed that the present invention is not restricted to any form of realization described previously and that some modifications can be added to the presented example of fabrication without reappraisal of the appended claims. For example, the present invention has been described referring to wheat and soybean leaves, but it is clear that the invention can be applied to any plant or crop leaf without departing from the scope of the invention.

1. An agrochemical composition comprising at least one agrochemical active component and a solvent of the formula (A): 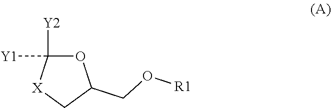
wherein
X is chosen from O or CH2;
Y1 and Y2 are independently of one another chosen from H or a linear or branched C1-C5 alkyl, or Y1 and Y2 together are a keto-group; and
R1 is chosen from a linear or branched C1-C20 alkyl.

2. The agrochemical composition according to claim 1, wherein X is CH2.

3. The agrochemical composition according to claim 1, wherein X is O.

4. The agrochemical composition according to claim 1, wherein Y1 and Y2 are independently of one another chosen from H or a linear or branched C1-C5 alkyl.

5. The agrochemical composition according to claim 1, wherein Y1 and Y2 together are a keto-group.

6. The agrochemical composition according to claim 1, wherein R1 is chosen from a linear or branched C1-C4 alkyl.

7. The agrochemical composition according to claim 1, wherein R1 is butyl, preferably R1 is n-butyl or tert-butyl, more preferably R1 is tert-butyl.

8. The agrochemical composition according to claim 1, wherein the agrochemical active component is chosen from the list of herbicides, fungicides, insecticides, algicides, molluscicides, miticides, rodenticides, plant growth regulators, biostimulants or a combination thereof.

9. The agrochemical composition according to claim 1, wherein said agrochemical active component is an herbicide chosen from the list of quizalofop-p-ethyl, diflufenican, phenmidipham, nicosulfuron, fluroxypyr and clethodim or a mixture thereof.

10. The agrochemical composition according to claim 1, wherein said agrochemical active component is a fungicide chosen from the list of azoxystrobin, metconazole, difenoconazole, tebuconazole, prothioconazole and spiroxamine or a mixture thereof.

11. The agrochemical composition according to claim 1, wherein said agrochemical active component is an insecticide chosen from the list of pyriproxyfen, imidacloprid, cypermethrin, chlorantraniliprole or a mixture thereof.

12. The agrochemical composition according to claim 1, wherein said agrochemical composition comprises said solvent in an amount of at least 3 to 80% by weight.

13. The agrochemical composition according to claim 1, wherein the solvent of the formula (A) has a solubility in water at 20° C. of at least 5.0% by weight.

14. The agrochemical composition according to claim 1, wherein the solvent of the formula (A) has an adjuvant property.

15. A method for applying an agrochemical composition according to claim 1 to an agricultural substrate, comprising the steps of:
emulsifying said agrochemical composition with water to provide an agrochemically applicable liquid, and
bringing said agrochemically applicable liquid in contact with said agricultural substrate.
